(19) United States
(12) Patent Application Publication
(10) Pub. No.: US 2016/0106565 A1
(43) Pub. Date: Apr. 21, 2016

(54) PANCREATICOBILIARY DIVERSION DEVICE

(71) Applicant: ENDOBETIX LTD., Misgav (IL)

(72) Inventors: Pierre SHARVIT, Hefer (IL); Chen PORAT, Kiryat Tivon (IL); Shlomo LEWKOWICZ, Tivon (IL); Tanhum FELD, Moshav Merhavya (IL)

(73) Assignee: ENDOBETIX LTD., Misgav (IL)

(21) Appl. No.: 14/896,582

(22) PCT Filed: Jun. 5, 2014
(86) PCT No.: PCT/IL14/50510

§ 371 Date: Dec. 7, 2015

Related U.S. Application Data
(60) Provisional application No. 61/832,195, filed on Jun. 7, 2013.

(51) Int. Cl. A61F 5/00

(52) U.S. Cl. CPC A61F 5/0076; A61F 5/0079; A61F 5/0033; A61F 5/0036; A61F 2002/045

(57) ABSTRACT

Apparatus (4020) comprising a pancreaticobiliary secretion-diversion guide (4030) for use with secretions that enter a gastrointestinal tract at an anatomical entry location. Guide (4030) comprises first and second anchors (4252/4054) which apply pressure to the wall of the tract. A resilient connecting element (2037) is coupled to the anchors. A collection-facilitation tube (2080) is between the anchors and in contact with and around element (2037). Tube (2080) has an inner surface (2045) and an outer surface (2046), the inner surface defining a lumen for passage of food. The outer surface inhibits contact of the secretions with food and defines a space (7) between the outer surface and the tract. A diversion tube (2090) is in fluid communication with the space and passes secretions from the space distally. An intragastric anchor (4152) coupled to the guide maintains the guide in place. Other applications are also described.

### CROSS-REFERENCES TO RELATED APPLICATIONS

The present application claims the priority of U.S. Provisional Application 61/832,195 to Sharvit et al., entitled, “Pancreaticobiliary diversion device,” filed on Jun. 7, 2013, which is incorporated herein by reference.

The present application is related to:

PCT Application PCT/IL2013/050054 to Sharvit et al., which published as WO/2013/108258 entitled, “Pancreaticobiliary diversion device,” filed on Jan. 17, 2013, which is incorporated herein by reference.

PCT Application PCT/IL2011/000579 to Sharvit et al., which published as WO/2012/011105 entitled, “Pancreatiobiliary diversion device,” filed on Jul. 20, 2011, which is incorporated herein by reference.

U.S. application Ser. No. 13/811,333 to Sharvit et al., which is a US national phase of PCT Application PCT/IL2011/000579, and published as US 2013-0197421 entitled “Pancreaticobiliary diversion device,” which is incorporated herein by reference.

U.S. Provisional Application 61/588,371 to Sharvit et al., entitled, “Pancreatiobiliary diversion device,” filed on Jan. 19, 2012, which is incorporated herein by reference.

U.S. Provisional Application 61/366,586 to Sharvit et al., entitled, “Pancreatiobiliary diversion device,” filed on Jul. 22, 2010, which is incorporated herein by reference.

### FIELD OF THE APPLICATION

Embodiments of the present invention relate generally to treatment of obesity and other conditions and particularly to treatment of obesity and other conditions by diversion of endogenous secretions.

### BACKGROUND OF THE APPLICATION

The human gastrointestinal tract is a system by which ingested food is digested and absorbed in order to provide the body with essential nutrients. The human gastrointestinal tract includes the small intestine, which is the longest portion of the digestive tract. The small intestine has three sections: the duodenum, jejunum and ileum. The duodenum, where most chemical digestion takes place, precedes the jejunum and ileum and is the shortest part (typically 25-30 cm in length) of the small intestine. The duodenum begins with the duodenal bulb and ends at the ligament of Treitz.

The digestion process is regulated by several hormones, some of which are released by the gastrointestinal tract. Additionally, many digestive enzymes are secreted by the gastrointestinal tract and the pancreas to aid in the digestion of food. Other endogenous secretions, such as bile, facilitate the digestion of lipids in the small intestine. Bile is typically stored in the gallbladder and upon eating is discharged into the duodenum.

Obesity and type II diabetes are serious health concerns. It is believed that obesity promotes insulin resistance, and has been found to play an important role in the pathogenesis of diabetes. Accordingly, weight loss is generally recommended, in order to lower elevated blood glucose levels in overweight and obese individuals with type II diabetes.

Some weight loss surgical techniques currently include several types of bariatric surgical procedures, including malabsorptive procedures, e.g., biliopancreatic diversion and biliopancreatic diversion with a duodenal switch. Generally, these diversion procedures, although they also reduce stomach size, are based mainly on creating malabsorption by bypassing digestion in the duodenum and other parts of the small intestine.

PCT Publication WO 2012/011105 to Sharvit et al., describes apparatus for use with pancreaticobiliary secretions that enter a gastrointestinal tract of a subject at an anatomical entry location, the apparatus including: a pancreaticobiliary secretion-diversion guide configured to collect the pancreaticobiliary secretions from the anatomical entry location and deliver the pancreaticobiliary secretions to a location in the gastrointestinal tract that is distal to the anatomical entry location; and an anchoring system. The anchoring system comprises one or more helical anchors located within the gastrointestinal tract and configured to apply pressure to a wall of the gastrointestinal tract in order to maintain the pancreaticobiliary secretion-diversion guide in place.

### SUMMARY OF APPLICATIONS

In some applications of the present invention, methods and apparatus are provided for diversion of pancreaticobiliary secretions that are secreted from a duodenal papilla of a subject, to a location in the gastrointestinal tract that is distal to the duodenal papilla.

For some applications, first and second anchors, e.g., stent anchors, are respectively coupled to proximal and distal portions of a pancreaticobiliary secretion-diversion guide that is disposed within a gastrointestinal tract. At least one resilient connecting element is coupled to the first and second anchors and together with the anchors, is configured to reduce motion of the pancreaticobiliary secretion-diversion guide within the gastrointestinal tract. The resilient connecting element is typically straight, in the absence of any forces applied thereto, and has a maximum diameter of 0.3- 1.5 mm. Alternatively, the resilient connecting element is curved, in the absence of any forces applied thereto.

A collection-facilitation tube is typically disposed around and in contact with the resilient connecting element and is located between the first and second anchors. The collection facilitation tube is typically flexible and, if the resilient connecting element is curved, the collection facilitation tube assumes a curved configuration when disposed around the curved resilient connecting element. The collection-facilitation tube has an inner surface and an outer surface, the inner surface defining a lumen for passage of food therethrough. The outer surface is disposed to inhibit contact of the pancreaticobiliary secretions with food within the lumen and is configured to define a space between the outer surface and the gastrointestinal tract for collecting the secretions. Typically, inhibiting association of the pancreaticobiliary secretions with ingested food reduces emulsification and formation of micelles of ingested fat, and disrupts the process of fat digestion in the body.

Optionally, the resilient connecting wire presses the collection-facilitation tube against the wall of the gastrointestinal tract, by the resilient connecting element contacting and applying pressure to the inner surface of the collection-facilitation tube.

The guide further comprises a diversion tube which is configured to be in fluid communication with the space between the outer surface of the collection-facilitation tube and the gastrointestinal tract. The diversion tube passes pancreaticobiliary secretions from the space between the outer surface of the collection-facilitation tube and the gastrointestinal tract, to a location in the gastrointestinal tract that is distal to an anatomical entry location (e.g., the papilla). The secretions are typically diverted to a location which is beyond the duodenum, e.g., beyond the ligament of Treitz which is the final section of the duodenum. The tube additionally inhibits contact of the pancreaticobiliary secretions with food that has passed through the lumen.

The apparatus typically further comprises an intragastric anchor coupled to the pancreaticobiliary secretion-diversion guide, and configured to be located in a stomach of the subject and to reduce motion, e.g., migration, of the pancreaticobiliary secretion-diversion guide within the gastrointestinal tract. For some applications, the first anchor, or portions thereof, are disposed in the stomach. For such applications. the apparatus does not comprise an additional intragastric anchor.

The inventors have identified that providing proper anchoring and reducing migration of a pancreaticobiliary secretion-diversion guide are of particular importance when deploying such a secretion-diversion guide within the gastrointestinal tract. As provided by some applications of the present invention, separate first and second stent anchors are connected by a resilient wire to facilitate anchoring of the secretion-diversion guide within the gastrointestinal tract. The inventors hypothesize that providing separate stent anchors which are connected by a resilient connecting element generally increases stability of the secretion-diversion guide in the gastrointestinal tract. This configuration allows for a given stent anchor to remain generally stationary while the other stent anchor is undergoing a distally-directed force due to a peristaltic wave applied to the other stent anchor. The stationary anchor thereby stabilizes the other stent anchor via the resilient connecting element (regardless of whether at a given point in time the stationary anchor is the more distal or more proximal of the two stent anchors.)

Additionally. the inventors hypothesize that providing separate stent anchors connected by a resilient connector, in contrast to a single elongated stent anchor, increases stability and reduces motion e.g., migration, of the secretion-diversion guide distally in the gastrointestinal tract due to an additional mechanism of operation. In the case of a single elongated stent anchor, a peristaltic wave affecting a given portion of the anchor would be likely to affect other portions of the anchor, resulting in impaired anchoring. For example, a squeezing force due to a peristaltic wave applied to one end of the single stent anchor, would cause a reduction in diameter of the other end of the single stent anchor (and consequent reduced anchoring), due to the nature of a braided stent. Providing independent stent anchors connected by a resilient connecting element, in accordance with some applications of the present invention, typically overcomes this problem. For example, a peristaltic wave applied to one stent anchor typically will not affect, e.g., cause squeezing reduction in diameter, of the other stent anchor.

There is therefore provided in accordance with some applications of the present invention, apparatus for use with pancreaticobiliary secretions that enter a gastrointestinal tract of a subject at an anatomical entry location the apparatus including:

a pancreaticobiliary secretion-diversion guide, including:
a proximal portion;
a distal portion;
first and second anchors coupled to the proximal and distal portions respectively and configured to apply pressure to the wall of the gastrointestinal tract in order to reduce motion of the pancreaticobiliary secretion-diversion guide within the gastrointestinal tract;
a resilient connecting element coupled to the first and second anchors and having a maximum diameter of 0.3- 1.5 mm;
a collection-facilitation tube between the first and second anchors and disposed in contact with and around the resilient connecting element, the collection-facilitation tube having an inner surface and an outer surface, the inner surface defining a lumen for passage of food therethrough, the outer surface disposed to inhibit contact of the secretions with food within the lumen and configured to define a space between the outer surface and the gastrointestinal tract for collecting the secretions;
a diversion tube, configured to:
   be in fluid communication with the space between the outer surface and the gastrointestinal tract,
   pass pancreaticobiliary secretions from the space to a location in the gastrointestinal tract that is distal to the anatomical entry location, and
   inhibit contact of the pancreaticobiliary secretions with food that has passed through the lumen; and

an intragastric anchor coupled to the pancreaticobiliary secretion-diversion guide, and configured to be located in a stomach of the subject and to reduce motion of the pancreaticobiliary secretion-diversion guide within the gastrointestinal tract.

For some applications, the first anchor is coupled to the intragastric anchor.

For some applications, the resilient connecting element is curved, in the absence of any forces applied thereto.

For some applications, the anatomical entry location includes a duodenal papilla of the subject, and the pancreaticobiliary secretion-diversion guide is configured to collect the pancreaticobiliary secretions that are secreted from the duodenal papilla.

For some applications, the pancreaticobiliary secretion-diversion guide is configured to collect the pancreaticobiliary secretions that are secreted from the duodenal papilla without entering a duct through which the secretions pass.

For some applications, the first and second anchors include stent anchors.

For some applications, the intragastric anchor includes a stent anchor.

For some applications, the second anchor has a diameter of 20-40 mm when unconstrained.

For some applications, the second anchor has a constant diameter along at least 80% of a longitudinal axis thereof.
For some applications, the intragastric anchor has a diameter of 45-65 mm when unconstrained.

For some applications, the first anchor has a diameter of 20-40 mm when unconstrained.

For some applications, the first anchor has a diameter of 30-40 mm when unconstrained.

For some applications, the diversion tube has a diameter of 4-20 mm.

For some applications, the collection-facilitation tube has a diameter of 20-25 mm.

For some applications, the resilient connecting element has a length of 10-18 cm when unconstrained, measured along a longitudinal axis of the resilient connecting element.

For some applications, the resilient connecting element has a length of 12-15 cm when unconstrained, measured along the longitudinal axis of the resilient connecting element.

There is further provided in accordance with some applications of the present invention apparatus for use with pancreaticobiliary secretions that enter a gastrointestinal tract of a subject at an anatomical entry location, the apparatus including:

a pancreaticobiliary secretion-diversion guide, including:
first and second stent anchors coupled to proximal and distal portions of the pancreaticobiliary secretion-diversion guide respectively, and configured to apply pressure to the wall of the gastrointestinal tract in order to reduce motion of the pancreaticobiliary secretion-diversion guide within the gastrointestinal tract, the first stent anchor including a proximal intragastric portion and a distal pyloric portion, the proximal intragastric portion (a) having a maximum unconstrained diameter of 45-65 mm, when no forces are applied thereto, and (b) being configured to diverge in a distal to proximal direction to a maximum diverged diameter of 50-60 mm, when a squeezing force is applied to the distal pyloric portion;
at least one resilient connecting element, each coupled to the first and second stent anchors and having a maximum diameter of 0.3- 1.5 mm;
a collection-facilitation tube between the first and second stent anchors and disposed in contact with and around the at least one resilient connecting element. the collection-facilitation tube having an inner surface and an outer surface, the inner surface defining a lumen for passage of food therethrough, the outer surface disposed to inhibit contact of the secretions with food within the lumen and configured to define a space between the outer surface and the gastrointestinal tract for collecting the secretions;
a diversion tube, configured to:
   be in fluid communication with the space between the outer surface and the gastrointestinal tract,
   pass pancreaticobiliary secretions from the space to a location in the gastrointestinal tract that is distal to the anatomical entry location, and
inhibit contact of the pancreaticobiliary secretions with food that has passed through the lumen.

For some applications, the first and second stent anchors are arranged such that application of a squeezing force to the second stent anchor that reduces a diameter thereof by 50% does not produce an increase in a maximum diameter of the proximal intragastric portion of more than 10%.

For some applications, the first stent anchor is arranged such that application of a squeezing force to the distal pyloric portion that reduces a diameter thereof by 50% causes an increase in the maximum diverged diameter the proximal intragastric portion by at least 1%

For some applications, the first and second stent anchors are arranged such that application of a squeezing force to the second stent anchor that reduces a diameter thereof by 50% does not produce an increase in a maximum diameter of the proximal intragastric portion.

For some applications, the second anchor has a diameter of 20-40 mm when unconstrained.

For some applications, the pyloric portion of the first anchor has a diameter of 30- 40 mm when unconstrained.

For some applications, the pyloric portion of the first anchor has a length of 10-30 mm when unconstrained, measured along a longitudinal axis of the anchor.

For some applications, the second anchor has a length of 40-60 mm when unconstrained, measured along a longitudinal axis of the anchor.

For some applications, the first anchor has a diameter of 20-40 mm when unconstrained.

For some applications, the intragastric portion of the first anchor has a length of 40-60 mm when unconstrained, measured along a longitudinal axis of the anchor.

For some applications, the diversion tube has a diameter of 4-20 mm.

For some applications, the collection-facilitation tube has a diameter of 20-25 mm.

For some applications, the anatomical entry location includes a duodenal papilla of the subject, and the pancreaticobiliary secretion-diversion guide is configured to collect the pancreaticobiliary secretions that are secreted from the duodenal papilla.

For some applications, the pancreaticobiliary secretion-diversion guide is configured to collect the pancreaticobiliary secretions that are secreted from the duodenal papilla without entering a duct through which the secretions pass.

For some applications, the at least one resilient connecting element includes at least two resilient connecting elements.

For some applications, the at least one resilient connecting element includes exactly two resilient connecting elements.

For some applications, the at least one resilient connecting element include not more than eight resilient connecting elements.

For some applications, each resilient connecting element has a length of 10-18 cm when unconstrained, measured along a longitudinal axis of the resilient connecting element.

For some applications, each resilient connecting element has a length of 12-15 cm when unconstrained, measured along the longitudinal axis of the resilient connecting element.

There is further provided in accordance with some applications of the present invention apparatus for use with pancreaticobiliary secretions that enter a gastrointestinal tract of a subject at an anatomical entry location, the apparatus including:

a pancreaticobiliary secretion-diversion guide, including:
an intragastric stent anchor coupled to a proximal portion of the pancreaticobiliary secretion-diversion guide and having a proximal portion and a distal portion and configured to reduce motion of the pancreaticobiliary secretion-diversion guide within the gastrointestinal tract;
an intestinal stent anchor coupled to a distal portion of the pancreaticobiliary secretion-diversion guide and configured to apply pressure to the wall of the gastrointestinal tract in order to reduce motion of the pancreaticobiliary secretion-diversion guide within the gastrointestinal tract,
at least one resilient connecting element, each coupled to the first and second stent anchors and having a maximum diameter of 0.3- 1.5 mm;
a collection-facilitation tube between the first and second stent anchors and disposed in contact with and around the at least one resilient connecting element, the collection-facilitation tube having an inner surface and an outer surface, the inner surface defining a lumen for passage of food therethrough, the outer surface disposed to inhibit contact of the secretions with food within the lumen and configured to define a space between the outer surface and the gastrointestinal tract for collecting the secretions;
a diversion tube, configured to:
   be in fluid communication with the space between the outer surface and the gastrointestinal tract,
   pass pancreaticobiliary secretions from the space to a location in the gastrointestinal tract that is distal to the anatomical entry location, and
inhibit contact of the pancreaticobiliary secretions with food that has passed through the lumen.

For some applications, a diameter of the proximal portion of the intragastric stent anchor is larger than a diameter of the distal portion of the intragastric stent anchor.

For some applications, the proximal portion of the intragastric stent anchor has a diameter of 45-65 mm, and the distal portion of the intragastric stent anchor has a diameter of 35-55 mm.

For some applications, a diameter of the proximal portion of the intragastric stent anchor is the same as a diameter of the distal portion of the intragastric stent anchor.

For some applications, the proximal portion of the intragastric stent anchor has a diameter of 45-65 mm.

For some applications, the intestinal stent has a constant diameter along at least 80% of a longitudinal axis thereof.

For some applications, the intestinal stent has a diameter of 20-40 mm along at least 80% of a longitudinal axis thereof.

For some applications, a ratio between a diameter of the proximal portion of the intragastric stent anchor and a diameter of the distal portion of the intragastric stent anchor is 1.1-1.8.

For some applications, a ratio between a diameter of the proximal portion of the intragastric stent anchor and a diameter of the distal portion of the intragastric stent anchor is 1-1.1.

For some applications, the intragastric stent anchor has a length of 40-60 mm when unconstrained, measured along a longitudinal axis of the anchor.

For some applications, the intragastric stent anchor is configured to be deployed entirely in a stomach without passing through a pylorus of the subject.

For some applications, the anatomical entry location includes a duodenal papilla of the subject, and the pancreaticobiliary secretion-diversion guide is configured to collect the pancreaticobiliary secretions that are secreted from the duodenal papilla.

For some applications, the pancreaticobiliary secretion-diversion guide is configured to collect the pancreaticobiliary secretions that are secreted from the duodenal papilla without entering a duct through which the secretions pass.

For some applications, the at least one resilient connecting element include not more than eight resilient connecting elements.

For some applications, the at least one resilient connecting element includes at least two resilient connecting elements.

For some applications, the at least one resilient connecting element includes exactly least two resilient connecting elements.

For some applications, each resilient connecting element has a length of 10-18 cm when unconstrained, measured along a longitudinal axis of the resilient connecting element.

For some applications, each resilient connecting element has a length of 12-15 cm when unconstrained, measured along a longitudinal axis of the resilient connecting element.

For some applications, the diversion tube has a diameter of 4-20 mm.

For some applications, the collection-facilitation tube has a diameter of 20-25 mm.

For some applications, the intestinal anchor is configured to be located downstream of the anatomical entry location of the pancreaticobiliary secretions.

There is further provided oin accordance with some application of the present invention, apparatus for use with pancreaticobiliary secretions that enter a gastrointestinal tract of a subject at an anatomical entry location the apparatus including:

a pancreaticobiliary secretion-diversion guide, including:
a proximal portion;
a distal portion;
first and second anchors coupled to the proximal and distal portions respectively and configured to apply pressure to the wall of the gastrointestinal tract in order to reduce motion of the pancreaticobiliary secretion-diversion guide within the gastrointestinal tract;
a curved resilient connecting element coupled to the first and second anchors and having a maximum diameter of 0.3- 1.5 mm
a collection-facilitation tube between the first and second anchors and disposed in contact with and around the curved resilient connecting element so as to assume a curved configuration, the collection-facilitation tube having an inner surface and an outer surface, the inner surface defining a lumen for passage of food therethrough, the outer surface disposed to inhibit contact of the secretions with food within the lumen and configured to define a space between the outer surface and the gastrointestinal tract for collecting the secretions;
a diversion tube, configured to:
   be in fluid communication with the space between the outer surface and the gastrointestinal tract,
   pass pancreaticobiliary secretions from the space to a location in the gastrointestinal tract that is distal to the anatomical entry location, and
   inhibit contact of the pancreaticobiliary secretions with food that has passed through the lumen; and

an intragastric anchor coupled to the pancreaticobiliary secretion-diversion guide, and configured to be located in a stomach of the subject and to reduce motion of the pancreaticobiliary secretion-diversion guide within the gastrointestinal tract,
the curved resilient connecting element is configured to reduce motion of the pancreaticobiliary secretion-diversion guide within the gastrointestinal tract by pressing the collection-facilitation tube against the wall of the gastrointestinal tract, by the resilient connecting element contacting and applying pressure to the inner surface of the collection-facilitation tube.

For some applications, the anatomical entry location includes a duodenal papilla of the subject, and the pancreaticobiliary secretion-diversion guide is configured to collect the pancreaticobiliary secretions that are secreted from the duodenal papilla.

For some applications, the pancreaticobiliary secretion-diversion guide is configured to collect the pancreaticobiliary secretions that are secreted from the duodenal papilla without entering a duct through which the secretions pass.

For some applications, the resilient connecting element is curved, in the absence of any forces applied thereto.

For some applications, the apparatus includes one or more space-occupying elements coupled to an inflation tube and disposed within the collection-facilitation tube.

For some applications, the space-occupying elements are inflatable.

For some applications, the anchors include stent anchors.

For some applications, each stent anchor has a diameter of 20-40 mm.

The present invention will be more fully understood from the following detailed description of embodiments thereof, taken together with the drawings, in which:

### BRIEF DESCRIPTION OF THE DRAWINGS

FIG. 1 is a schematic illustration of apparatus for diversion of pancreaticobiliary secretions, in accordance with some applications of the present invention;

FIG. 2 is a schematic illustration of the apparatus for diversion of pancreaticobiliary secretions, in accordance with some applications of the present invention;

FIG. 3 is a schematic illustration of the apparatus for diversion of pancreaticobiliary secretions further including one or more space-occupying elements, in accordance with some applications of the present invention;

FIG. 4 is a schematic illustration of apparatus for diversion of pancreaticobiliary secretions, in accordance with some applications of the present invention;

FIGS. 5A-C are schematic illustrations of apparatus for diversion of pancreaticobiliary secretions, in accordance with some applications of the present invention;

FIGS. 6A-B are schematic illustrations of the apparatus for diversion of pancreaticobiliary secretions shown in FIGS. 5A-C, showing an alternative configuration for the pyloric portion of the proximal anchor, in accordance with some applications of the present invention;

FIG. 7 is a schematic illustrations of apparatus for diversion of pancreaticobiliary secretions, in accordance with some applications of the present invention; and

FIGS. 8A-B are reproductions of x-ray images acquired during experiments performed in accordance with some applications of the present invention.

### DETAILED DESCRIPTION OF APPLICATIONS

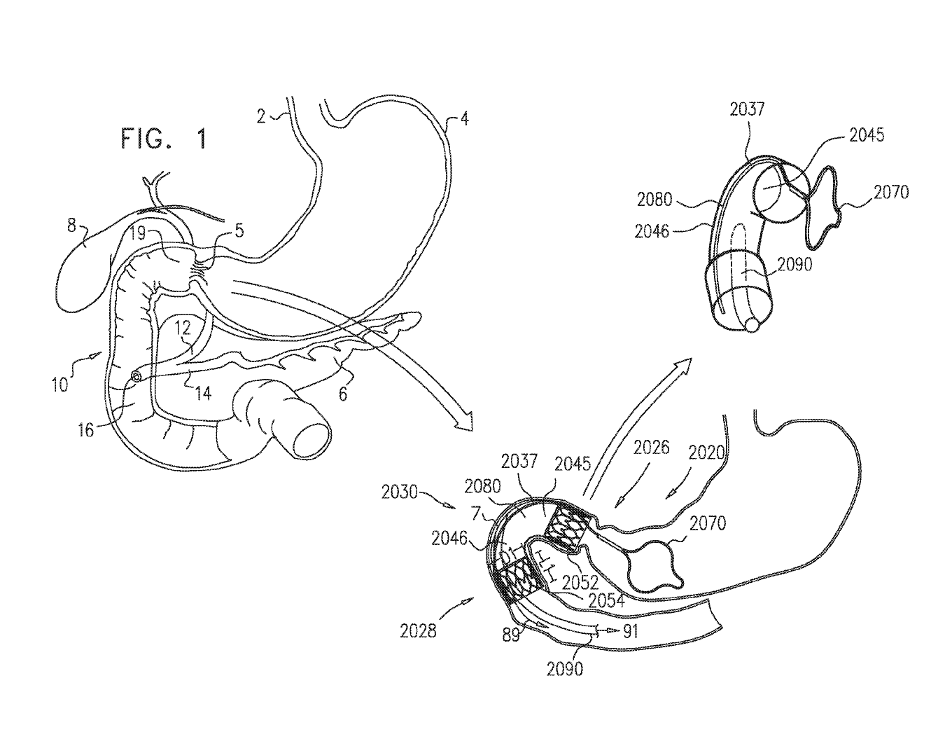

Reference is made to FIG. 1 which is a schematic illustration of apparatus 2020 for diversion of pancreaticobiliary secretions, in accordance with some applications of the present invention. Apparatus 2020 is typically configured for placement inside a gastrointestinal tract of a subject. FIG. 1 provides a schematic illustration of several components of a human digestive system. During the process of food digestion, food passes through esophagus 2 into stomach 4. The content of stomach 4 passes through pylorus 5 into the first section of the small intestine, duodenum 10. Bile, which aids in the process of fat digestion, is stored between meals in gallbladder 8. When the bile is released from gallbladder 8, it flows through the cystic duct and the common bile duct 12. Pancreas 6 produces exocrine secretions, including digestive enzymes, which pass through pancreatic duct 14. Pancreatic duct 14 merges with common bile duct 12 and together they form, at a medial side of a second portion of duodenum 10, a structure called the major duodenal papilla 16. Thus, major duodenal papilla 16 is an anatomical entry location of pancreaticobiliary secretions into the gastrointestinal tract. In some cases, common bile duct 12 discharges into the duodenum through a papilla which is in close proximity to major duodenal papilla 16. It will be appreciated that some applications of the present are applicable to such cases as well.

Some applications of the present invention comprise apparatus 2020 which comprises a pancreaticobiliary secretion-diversion guide 2030. Guide 2030 is inserted into the gastrointestinal tract of the subject and collects pancreaticobiliary secretions from duodenal papilla 16. Guide 2030 typically delivers the pancreaticobiliary secretions to a location in the gastrointestinal tract that is distal to the duodenal papilla. Typically, guide 2030 transfers the pancreaticobiliary secretions to a location that is beyond the duodenum, e.g., beyond the ligament of Treitz, which is the final section of the duodenum. For some applications, guide 2030 diverts the pancreaticobiliary secretions to a location that is at least 40 cm or less than 80 cm, e.g., between 40 and 80 cm beyond the ligament of Treitz. For some applications, guide 2030 is configured to divert the pancreaticobiliary secretions to any location within the small intestine that is distal to the ligament of Treitz (e.g., 100-120 cm distal to the ligament of Treitz).

Guide 2030 comprises a first anchor 2052 and a second anchor 2054 which are coupled to a proximal portion 2026 and a distal portion 2028 of guide 2030, respectively. In this context, in the specification and in the claims, “proximal” means closer to the orifice through which the guide is originally placed into the body, and “distal” means further from this orifice. Anchors 2052 and 2054 are typically disposed in duodenum 10 in a location that is in the vicinity of duodenal papilla 16. Anchors 2052 and 2054 each apply pressure to the wall of the gastrointestinal tract in order to reduce motion of pancreaticobiliary secretion-diversion guide 2030 within the intestine, even while the intestine undergoes peristalsis.

Typically, first anchor 2052 is disposed upstream of duodenal papilla 16 and second anchor 2054 is disposed downstream of duodenal papilla 16.

Anchors 2052 and 2054 are shaped to define an anchor lumen which allows passage of chyme, i.e., partly digested food, therethrough.

For some applications first anchor 2052 is deployed within the first section of duodenum 10, duodenal bulb 19. Anchor 2052 pushes against the walls of duodenal bulb 19, and applies pressure to the wall of duodenal bulb 19 in order to maintain pancreaticobiliary secretion-diversion guide 2030 in place.

As shown in FIG. 1, for some applications, first anchor 2052 and second anchor 2054 comprise stent anchors. Anchors 2052 and 2054 typically assume a diameter that is at least 20 mm, and/or less than 40 mm, e.g., between 20-40 mm when no force is applied thereto. When deployed within the gastrointestinal tract, anchors 2052 and 2054 typically assume a diameter D1 that is at least 20 mm and/or less than 35 mm, e.g., 30 mm. A resting length L1 of each of anchors 2052 and 2054 (i.e., if not constrained by the gastrointestinal tract) is typically 20-45 mm, e.g., at least 20 mm and/or less than 45 mm, e.g., 30 mm measured along a longitudinal axis of each stent anchor.

For some applications, anchors 2052 and 2054 are flexible. Typically, the flexibility of anchors 2052 and 2054 facilitates endoscopic passage of anchors 2052 and 2054 in a generally compressed state thereof through the esophagus. For some applications, anchors 2052 and 2054 comprise a shape memory material which is configured to expand within the gastrointestinal tract.

In addition to anchors 2052 and 2054, guide 2030 comprises a collection-facilitation tube 2080, located between the first and second anchors. Collection-facilitation tube 2080 has an inner surface 2045 and an outer surface 2046, the inner surface defining a lumen for passage of food therethrough (indicated by arrow 89), and the outer surface being disposed to inhibit contact of the secretions with chyme within the lumen. Outer surface 2046 is additionally configured to define a space 7 between outer surface 2046 and the gastrointestinal tract, for collecting pancreaticobiliary secretions. Collection-facilitation tube 2080 when fully open typically has a diameter of at least 15 mm and/or less than 30 mm, e.g., at least 20 mm and/or less than 25 mm. Collection-facilitation tube 2080 typically has a resting length of 60 mm to 150 mm.

Prior to guide 2030 being disposed in the intestine of the subject (e.g., when it is held straight, for example by an introducer or a rigid guidewire), a distance between the proximal edge of anchor 2052 and the distal edge of anchor 2054 is typically between 10- 25 cm, e.g., 15 cm. Correspondingly, the distance between the distal edge of anchor 2052 and the proximal edge of anchor 2054 is typically between 5-15 cm, e.g., 9 cm.

In addition to anchors 2052 and 2054 and collection-facilitation tube 2080. guide 2030 further comprises a resilient, connecting element 2037. Resilient connecting element 2037 is coupled to first and second anchors, 2052 and 2054, and is configured to reduce motion of guide 2030 within the gastrointestinal tract. Resilient connecting element 2037 typically supports anchors 2052 and 2054 during peristalsis and reduces the possibility that a peristaltic wave affecting one of the anchors will result in migration of guide 2030 distally in the intestine.

Optionally, element 2037 assumes a curved shape as shown in FIG. 1 when no external force is applied thereto. Prior to guide 2030 being placed in the intestine of the subject, element 2037 may be in a straight configuration (e.g., when it is held straight, for example by an introducer or a generally rigid guidewire). Additionally, as shown in FIG. 1, collection-facilitation tube 2080 is typically flexible and assumes a curved configuration when disposed around the curved resilient wire. For such applications, resilient connecting element 2037 reduces motion of guide 2030 within the gastrointestinal tract by pressing collection-facilitation tube 2080 against the wall of the gastrointestinal tract, by element 2037 contacting and applying pressure to inner surface 2045 of collection-facilitation tube 2080.

Optionally, connecting element 2037 extends distally beyond second anchor 2054. Element 2037 typically comprises blunt ends so as not to injure the wall of the intestine of the subject.

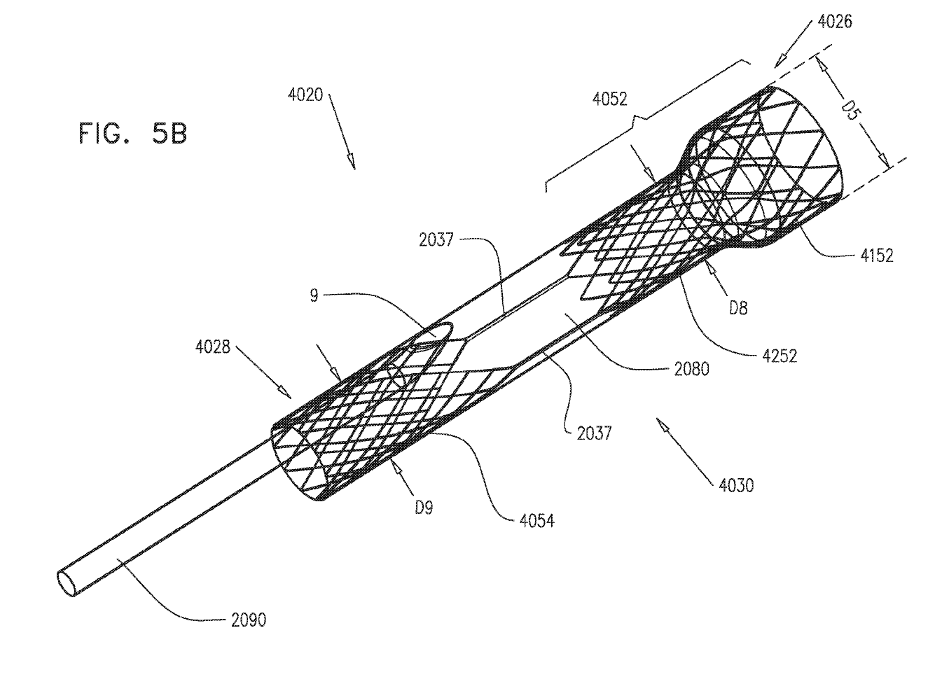

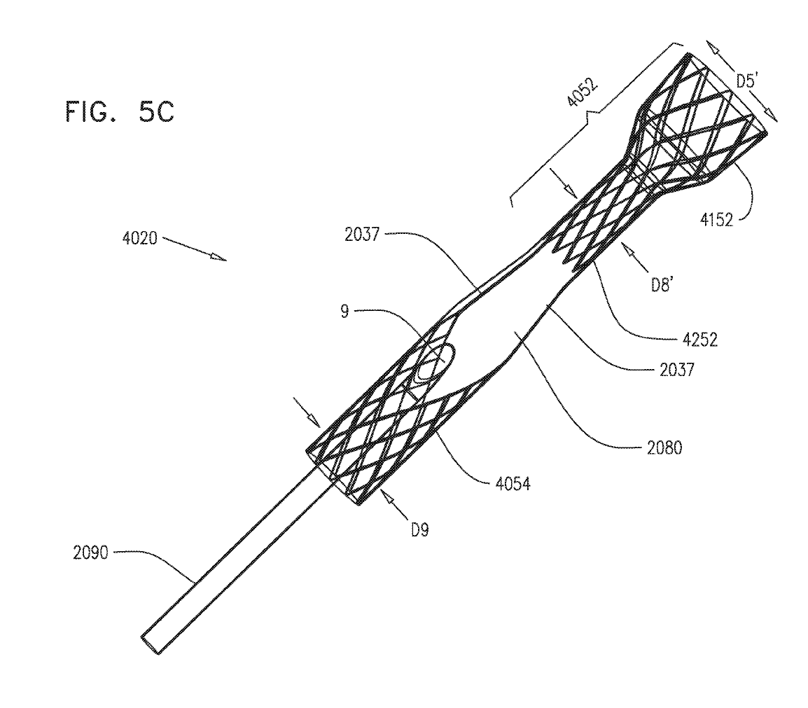

Pancreaticobiliary secretion-diversion guide 2030 further comprises a diversion tube 2090 which is configured to be in fluid communication with space 7 through an aperture 9 in collection-facilitation tube 2080, between outer surface 2046 and the gastrointestinal tract. Aperture 9 (shown in FIG. 5B) typically has a diameter of 7-15 mm. Diversion tube 2090 passes pancreaticobiliary secretions from space 7 to a location in the gastrointestinal tract that is distal to the anatomical entry location, and inhibits contact of the pancreaticobiliary secretions with food that has passed through the lumen of tube 2080. Diversion tube 2090 typically has a diameter of 4-20 mm, e.g., 5-15 mm, such as 10 mm. Arrow 91 indicates the direction of flow of pancreaticobiliary secretions in diversion tube 2090.

Guide 2030 is inserted into a gastrointestinal tract of a subject, and is typically disposed within duodenum 10 in a location that is in the vicinity of duodenal papilla 16, such that secretions entering the duodenum at papilla 16 are directly collected into diversion tube 2090 of guide 2030. The pancreaticobiliary secretions flow through tube 2090 of guide 2030 and are typically discharged from the guide in an area that is in the upper and/or mid jejunum. It is to be noted that for some applications guide 2030 and tube 2090 are configured (i.e., sufficient in length) to deliver the pancreaticobiliary secretions to a location that is in the lower jejunum or the ileum of the small intestine.

For some applications, a stiffening member is inserted into diversion tube 2090 to inhibit closing and possible entanglement of tube 2090 within the intestine. For example, a guide wire over which apparatus 2020 is deployed in the gastrointestinal tract remains in place within tube 2090 following deployment of the apparatus. For some applications, the stiffening member is coupled to an inner wall of tube 2090. For some applications, a small-diameter inflation tube is advanced through tube 2090 and inflated upon deployment of apparatus 2090 in order to maintain tube 2090 in an open configuration and inhibit closing and possible entanglement of tube 2090 within the intestine.

For some applications, diversion tube 2090 may include a braided mesh, in order to inhibit closing and possible entanglement of tube 2090 within the intestine.

Additionally or alternatively, the inner surface of tube 2090 is coated with a material that inhibits closing and possible entanglement of tube 2090 within the intestine.

For some applications, an inflatable element is coupled to a distal end of diversion tube 2090 in order to facilitate deployment of apparatus 2020 within the gastrointestinal tract (not shown). The inflatable element is typically easily advanced distally in the gastrointestinal tract until apparatus 2020 is properly deployed. Following deployment of apparatus 2020 the inflatable element is deflated and naturally passes from the body.

For some applications, the distal tip of diversion tube 2090 is closed, to facilitate tube 2090 being pushed distally by a pusher (e.g., a resilient wire). Typically, but not necessarily, the pusher is removed from the gastrointestinal tract following deployment of tube 2090. Holes formed in the lateral wall of tube 2090 (not shown) allow secretions passing through tube 2090 to exit the tube.

For some applications, apparatus 2020 further comprises an intragastric anchor 2070, which is configured for deployment within the stomach of the subject (e.g., within the pyloric antrum of the stomach). Intragastric anchor 2070 is coupled to guide 2030 and assists in maintaining guide 2030 in place within the gastrointestinal tract and to reduce motion of the pancreaticobiliary secretion-diversion guide within the gastrointestinal tract. Intragastric anchor 2070 typically inhibits migration of guide 2030 distally in the intestine.

Intragastric anchor 2070 is typically configured to remain within the stomach of the subject and accordingly is sufficient in size, and shaped in a manner, that prevents it from passing through the pylorus. Optionally, portions of intragastric anchor 2070 contact the wall of the stomach and may apply pressure to the wall of the stomach in order to maintain guide 2030 in place.

Intragastric anchor 2070 may have any suitable shape that is configured to remain within the stomach to facilitate anchoring of guide 2030 within the gastrointestinal tract of the subject. Typically, intragastric anchor 2070 has a longest diameter of at least 25 mm or less than 50 mm, e.g., between 25 and 50 mm, e.g., 40 mm.

Typically, resilient connecting element 2037 couples intragastric anchor 2070 to first anchor 2052 of guide 2030. It is noted that the connecting element does not interfere with proper functioning of the pylorus.

Retrieval of apparatus 2020 is typically done by attaching to anchor 2070 and removing apparatus 2020 from within the body of the subject.

For some applications, first anchor 2052 is disposed in stomach 4. For such applications, anchor 2052 is configured to remain within the stomach (typically in the pyloric antrum) and does not pass through the pylorus. Optionally, portions of first anchor 2052 contact the wall of the stomach and may apply pressure to the wall of the stomach in order to maintain guide 2030 in place. For such applications apparatus 2020 does not comprise intragastric anchor 2070.

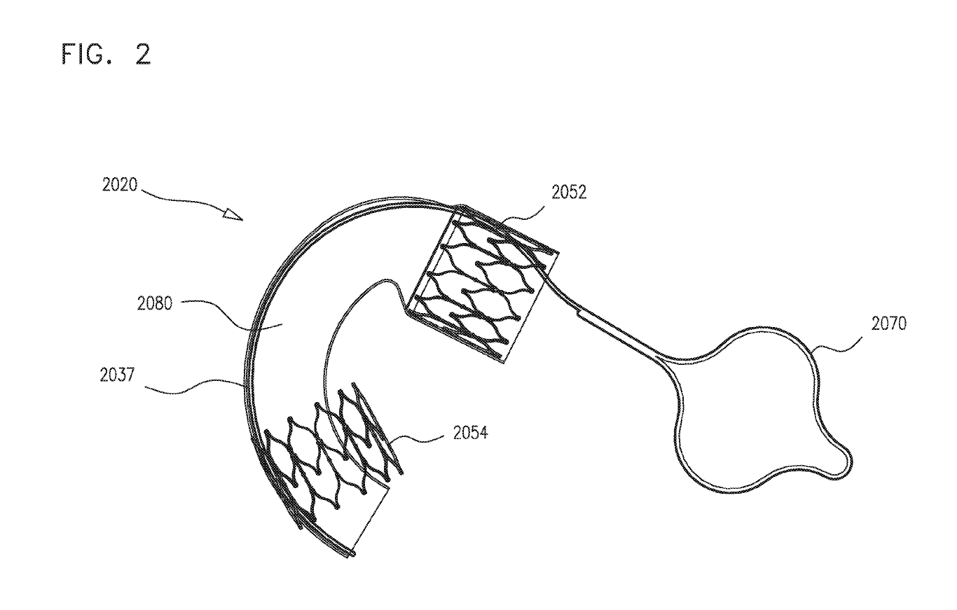

Reference is made to FIG. 2 which is a schematic illustration of apparatus 2020, in accordance with some applications of the present invention. As shown in FIG. 2, collection facilitation tube 2080 is disposed over first anchor 2052 and passes through second anchor 2054. For such applications, apparatus 2020 typically does not comprise diversion tube 2090 and pancreaticobiliary secretions flow distally in the intestine along outer surface 2046 of tube 2080. Outer surface 2046 of tube 2080 typically inhibits contact of the pancreaticobiliary secretions with chyme present within the lumen of collection-facilitation tube 2080. For such applications, collection-facilitation tube 2080 extends distally beyond second anchor 2054, in order to inhibit contact between chyme and pancreaticobiliary secretions. For example, collection-facilitation tube 2080 is sufficient in length to deliver the chyme to a location that is in the lower jejunum or the ileum of the small intestine.

It is noted that distal anchor 2054 is typically coated with a membrane to prevent injury to the intestine wall and ingrowth of tissue on the anchor.

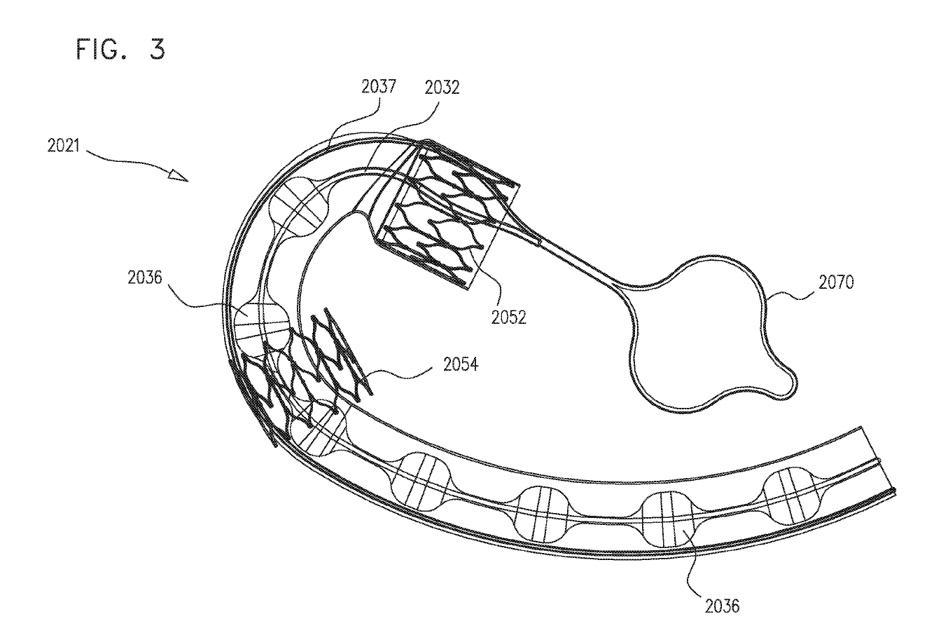

Reference is made to FIG. 3 which is a schematic illustration of apparatus 2021 for diversion of pancreaticobiliary secretions, in accordance with some applications of the present invention.

For some applications, one or more space-occupying elements 2036 are coupled to an inflation tube 2032. Typically, the space-occupying elements are configured to slow passage of chyme through the intestine and to press against the intestine wall in order to contribute to a sense of satiety following a meal. Space-occupying elements 2036 typically comprise inflatable elements that are inflated by inflation tube 2032 to a desired volume (by filling the inflatable element with fluid, i.e., a gas or a liquid).

Typically, inflation tube 2032 is coupled to a proximal portion of resilient connecting element 2037.

Reference is made to FIGS. 1-3. For some applications, apparatus 2020 and/or 2021 do not comprise second anchor 2054.

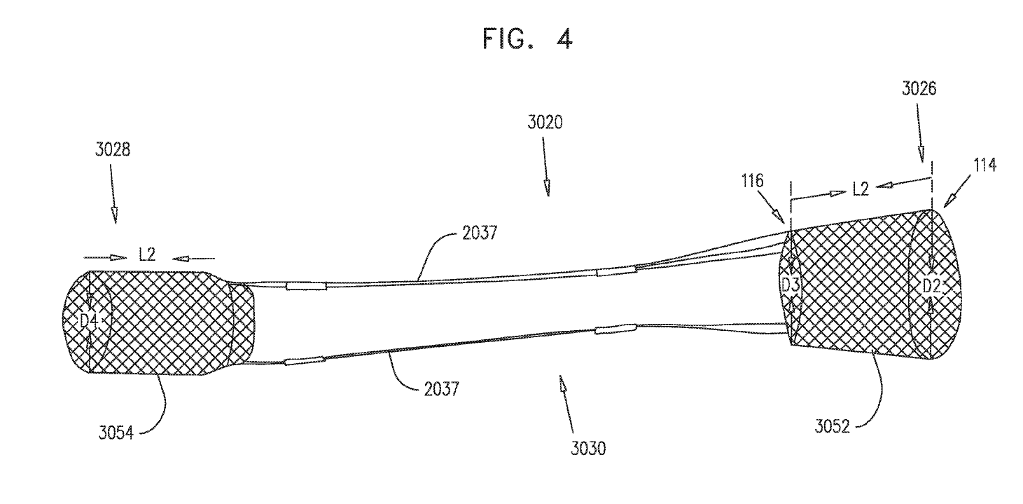

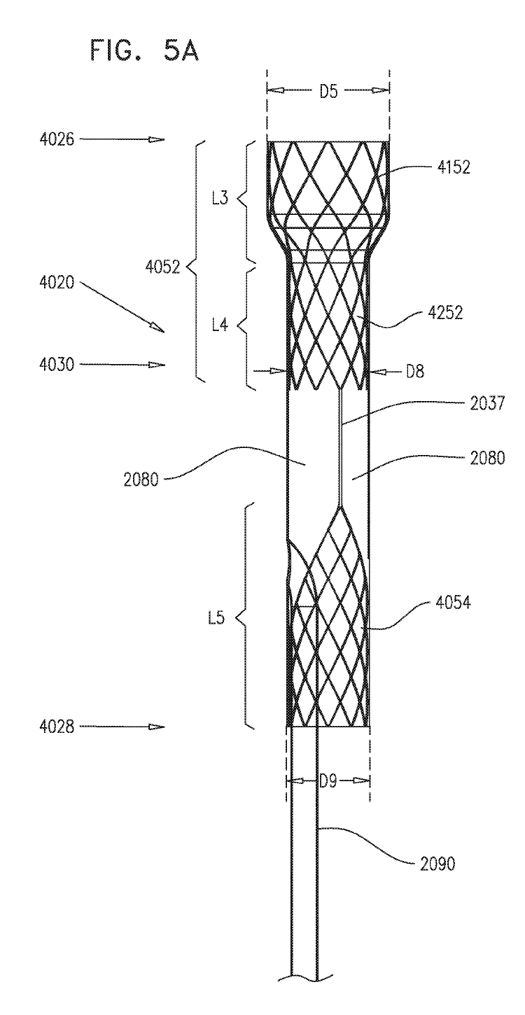

Reference is now made to FIGS. 4-7, which are schematic illustrations of additional possible configurations of the pancreaticobiliary secretion-diversion guide, as provided by some applications of the present invention. In particular, FIGS. 4-7 show alterative configurations for the first stent anchor of the pancreaticobiliary secretion-diversion guide. It is noted that unless stated otherwise, the apparatus shown in FIGS. 4-7 (apparatus 3020, 4020 and 5020) are the same as apparatus 2020 as described hereinabove with reference to FIG. 1. It is additionally noted that principles and features described hereinabove with reference to apparatus 2020 and guide 2030 apply to the apparatus illustrated in FIGS. 4-7. For example, the configuration of having separate first and second anchors connected by at least one resilient connecting element, to enhance anchoring and reduce motion, applies to the configurations shown in FIGS. 4-7.

Reference is first made to FIG. 4, which is a schematic illustration of apparatus 3020 for diversion of pancreaticobiliary secretions, in accordance with some applications of the present invention. Apparatus 3020 is typically configured for placement inside a gastrointestinal tract of a subject and is generally similar to apparatus 2020 except for differences described herein.

Typically, apparatus 3020 comprises a pancreaticobiliary secretion-diversion guide 3030. Guide 3030 is inserted into the gastrointestinal tract of the subject and collects pancreaticobiliary secretions from duodenal papilla 16 (shown in FIG. 1) and delivers the pancreaticobiliary secretions to a location in the gastrointestinal tract that is distal to the duodenal papilla, as described hereinabove with reference to apparatus 2020.

Guide 3030 typically comprises a first, intragastric stent anchor 3052 and a second intestinal stent anchor 3054, which are coupled to a proximal portion 3026 and a distal portion 3028 of guide 3030, respectively. For some applications, anchor 3052 is disposed entirely within the stomach, and anchor 3054 is disposed in the duodenum in a location that is typically downstream to the duodenal papilla.

Anchors 3052 and 3054 contact the wall of the gastrointestinal tract and typically reduce motion of pancreaticobiliary secretion-diversion guide 3030 within the gastrointestinal tract, even while the gastrointestinal tract undergoes peristalsis. Both anchors 3052 and 3054 are shaped to define an anchor lumen which allows passage of chyme, i.e., partly digested food, therethrough.

For some applications, anchor 3052 diverges in a distal to proximal direction to define a funnel-shaped anchor. As shown in FIG. 4, a proximal portion 114 of anchor 3052 has a diameter D2 that is larger than a diameter D3 of distal portion 116 of anchor 3052. A diameter D2 of proximal portion 114 is typically at least 45 mm, and/or less than 65 mm, e.g., between 45- 65 mm, e.g., 55 mm, when no force is applied to anchor 3052. A diameter D3 of distal portion 116 is typically at least 35 mm, and/or less than 55 mm, e.g., between 35-55 mm, e.g., 45 mm, when no force is applied to anchor 3052. The ratio of D2 to D3 is typically at least 1.1 and/or less than 1.8, e.g., between 1.1 and 1.8. For example, the ratio of D2 to D3 may be at least 1.2 and/or less than 1.5, e.g., between 1.2 and 1.5.

For other applications, first anchor 3052 has a generally constant diameter along at least 80% of a longitudinal axis thereof. For such applications, first anchor 3052 has a diameter of at least 40 mm, and/or less than 65 mm, e.g., between 40-65 mm, e.g., 55 mm, when no force is applied to anchor 3052. The ratio of D2 to D3 is typically at least 1 and/or less than 1.1, e.g., between 1 and 1.1.

Second anchor 3054 typically has a diameter D4 that is at least 20 mm, and/or less than 40 mm, e.g., between 20-40 mm when no force is applied thereto. When deployed within the gastrointestinal tract, anchor 3054 assumes a diameter that is at least 20 mm and/or less than 35 mm, e.g., 30 mm. Second anchor 3054 typically has a generally constant diameter along at least 80% of a longitudinal axis thereof.

A resting length L2 of anchor 3052 and anchor 3054 (i.e., if not constrained by the gastrointestinal tract) is typically at least 40 mm and/or less than 60 mm, e.g., between 40-60 mm, e.g., 50 mm measured along a longitudinal axis of anchor 3052. (The resting lengths of anchors 3052 and 3054 are not necessarily identical.)

Prior to guide 3030 being disposed in the gastrointestinal tract of the subject (e.g., when it is held straight, for example by an introducer or a rigid guidewire), a distance between the proximal edge of anchor 3052 and the distal edge of anchor 3054 is typically between 10-25 cm, e.g., 15-18 cm. Correspondingly, the distance between the distal edge of anchor 3052 and the proximal edge of anchor 3054 is typically between 5-15 cm, e.g., 9-15 cm, e.g., 12-15 cm.

In addition to anchors 3052 and 3054, guide 3030 further comprises at least one, e.g., two, resilient, connecting elements 2037. Each resilient connecting element 2037 is coupled to first and second anchors 3052 and 3054. Each element 2037 may be either straight or curved, in the absence of any forces applied thereto. Typically, but not necessarily, straight resilient connecting elements 2037 assume a curved configuration when disposed within the gastrointestinal tract, due to the local shape of the gastrointestinal tract. As described hereinabove with reference to FIG. 1, each resilient connecting element typically has a maximum diameter of 0.3-1.5 mm. It is noted that guide 3030 does not comprise more than eight connecting elements 2037. Typically, each resilient connecting element 2037 has a length of at least 100 mm and/or less than 180 mm, e.g., between 100-180 mm, for example, 120-150 mm.

Elements 2037 are configured, together with anchors 3052 and 3054, to reduce motion of guide 3030, within the gastrointestinal tract. Portions of anchors 3052 and 3054 typically contact and apply pressure to the wall of the gastrointestinal tract in order to stabilize guide 3030 and reduce motion of apparatus 3020. The gastrointestinal tract typically undergoes peristalsis and apparatus 3020 is subject to peristaltic waves which can cause migration of the guide 3030 distally along the gastrointestinal tract. Connecting elements 2037 typically provide support for anchors 3052 and 3054 during a peristaltic wave and thereby reduce motion of apparatus 3020. For example, when first anchor 3052 is subject to a peristaltic wave that applies a squeezing force to anchor 3052, guide 3030 remains generally stationary within the gastrointestinal tract because second anchor 3054 is stabilized by connecting elements 2037.

In addition to anchors 3052 and 3054 and connecting elements 2037, apparatus 3020 comprises a diversion lube 2090 and a collection-facilitation lube 2080 (not shown). Diversion tube 2090 and collection-facilitation tube 2080 are as described hereinabove with reference to FIG. 1.

Reference is now made to FIGS. 5A-C, which are views of apparatus 4020 for diversion of pancreaticobiliary secretions, in accordance with some applications of the present invention. Apparatus 4020 is typically configured for placement inside the gastrointestinal tract. Apparatus 4020 comprises a pancreaticobiliary secretion-diversion guide 4030. Guide 4030 is inserted into the gastrointestinal tract of the subject and collects pancreaticobiliary secretions from duodenal papilla 16 (shown in FIG. 1) and delivers the pancreaticobiliary secretions to a location in the gastrointestinal tract that is distal to the duodenal papilla, as described hereinabove with reference to apparatus 2020. It is noted that apparatus 4020 is generally similar to apparatus 2020 and 3020 except for differences described herein.

Pancreaticobiliary secretion-diversion guide 4030 typically comprises first and second stent anchors 4052 and 4054 which are coupled to proximal and distal portions 4026 and 4028 of guide 4030 respectively. Portions of anchors 4052 and 4054 apply pressure to the wall of the gastrointestinal tract in order to reduce motion of pancreaticobiliary secretion-diversion guide 4030 within the gastrointestinal tract.

First stent anchor 4052 typically comprises a proximal intragastric portion 4152 and a distal pyloric portion 4252. When deployed within the gastrointestinal tract, guide 4030 is positioned such that intragastric portion 4152 of first anchor 4052 is deployed in the stomach and pyloric portion 4252 of first anchor 4052 is deployed in the pylorus (and may also extend into the duodenum). Second anchor 4054 is deployed in the duodenum in a location that is typically downstream to the duodenal papilla.

Proximal intragastric portion 4152 typically has a maximum unconstrained diameter of D5 that is at least 45 mm, and/or less than 65 mm, e.g., between 45-65 mm, e.g., 55 mm, when no force is applied to anchor 4052. Intragastric portion 4152 diverges in a distal to proximal direction to a maximum diverged diameter D5′ of at least 50 mm, and/or less than 65 mm, e.g., between 50-65 mm, e.g., 60 mm, when squeezing forces are applied to distal pyloric portion 4252 (shown in FIG. 5C). For example, application of a squeezing force to distal pyloric portion 4252 that reduces the diameter thereof by 50% (from D8 to D8′) causes an increase in the maximum diverged diameter of the proximal intragastric portion 4152 by at least 1%, e.g., between 1-3% (from D5 to D5′). As shown in FIG. 5C, application of a squeezing force to pyloric portion 4252 generally does not cause a change in diameter D9 of second anchor 4054.

Distal pyloric portion 4252 typically has a diameter D8 of 30-40 mm, e.g., 35 mm.

A resting length L3 of intragastric portion 4152 of first anchor 4052 (i.e., if not constrained by the gastrointestinal tract) is typically at least 40 mm and/or less than 60 mm, e.g., between 40-60 mm, e.g., 50 mm, measured along a longitudinal axis of portion 4152.

A resting length L4 of pyloric portion 4252 of first anchor 4052 (i.e., if not constrained by the gastrointestinal tract) is typically at least 10 mm and/or less than 30 mm e.g., between 10-30 mm, e.g., 15-20 mm, measured along a longitudinal axis of portion 4252.

Second anchor 4054 typically has a diameter D9 that is at least 20 mm, and/or less than 40 mm, e.g., between 20-40 mm, when no force is applied thereto. When deployed within the gastrointestinal tract, anchor 4054 assumes a diameter that is at least 20 mm and/or less than 35 mm, e.g., 30 mm.

A resting length L5 of anchor 4054 (i.e., if not constrained by the gastrointestinal tract) is typically at least 40 mm and/or less than 60 mm, e.g., between 40-60 mm, e.g., 50 mm, measured along a longitudinal axis anchor 4054.

Prior to guide 4030 being disposed in the gastrointestinal tract of the subject (e.g., when it is held straight, for example by an introducer or a rigid guidewire), a distance between the proximal edge of anchor 4052 and the distal edge of anchor 4054 is typically between 10-25 cm, e.g., 15-18 cm. Correspondingly, the distance between the distal edge of anchor 4052 and the proximal edge of anchor 4054 is typically between 5-15 cm, e.g., 9-15 cm, e.g., 12-15 cm.

Apparatus 4020 typically further comprises at least one, e.g., two, resilient connecting elements 2037, each coupled to the first and second stent anchors. Connecting elements 2037 function as described hereinabove.

In addition to anchors 4052 and 4054 and connecting elements 2037, guide 4030 comprises a collection-facilitation tube 2080 and a diversion tube 2090. Diversion tube 2090 and collection-facilitation tube 2080 are as described hereinabove with reference to FIG. 1.

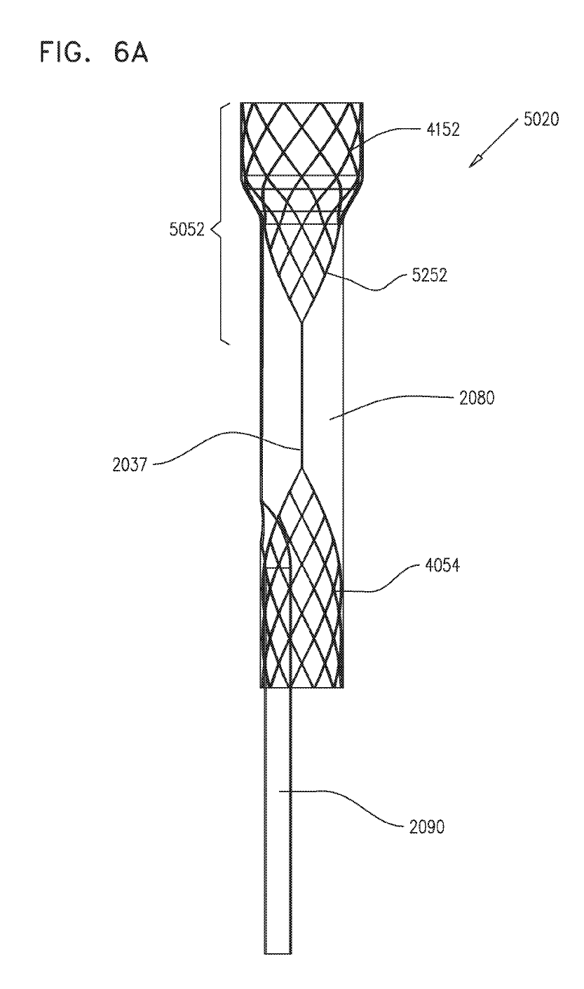

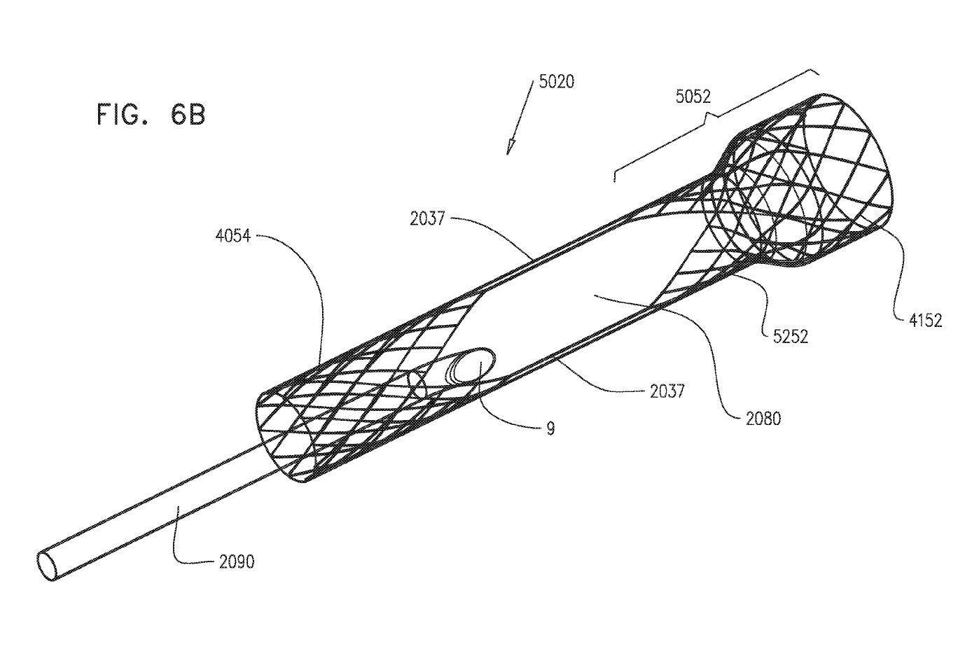

Reference is now made to FIGS. 6A-B, which are views of an additional configuration of a guide for diversion of pancreaticobiliary secretions, in accordance with some applications of the present invention. Apparatus 5020, shown in FIGS. 6A-B, is generally the same as apparatus 4020 shown in FIGS. 5A-B with the exception that distal pyloric portion 5252 of first anchor 5052 typically does not completely surround the lumen of collection-facilitation tube 2080. Other components of apparatus 5020, including proximal intragastric portion 4152 of a first anchor 5052, are as described with reference to apparatus 4020.

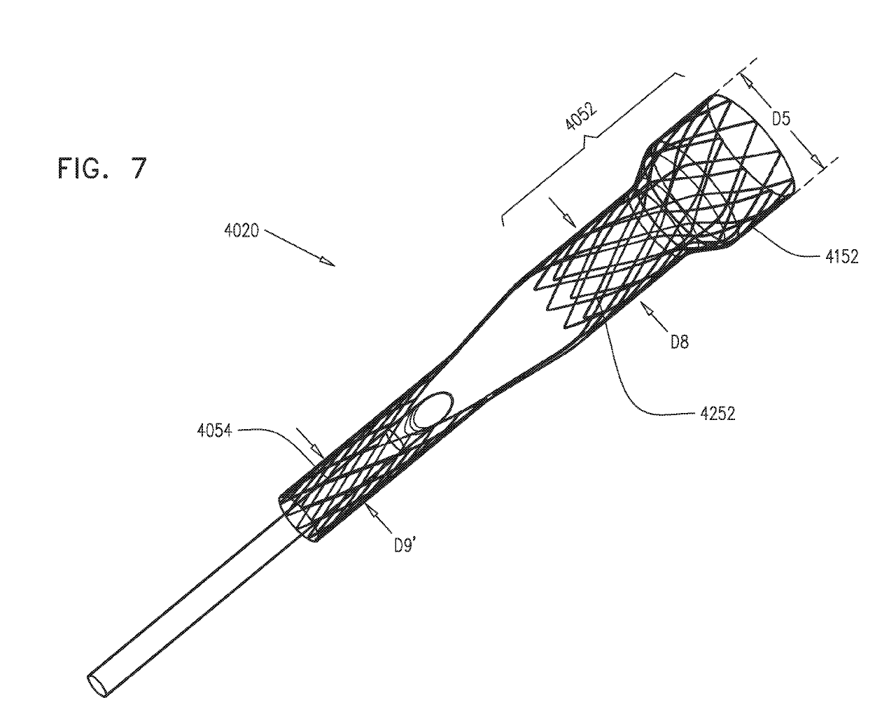

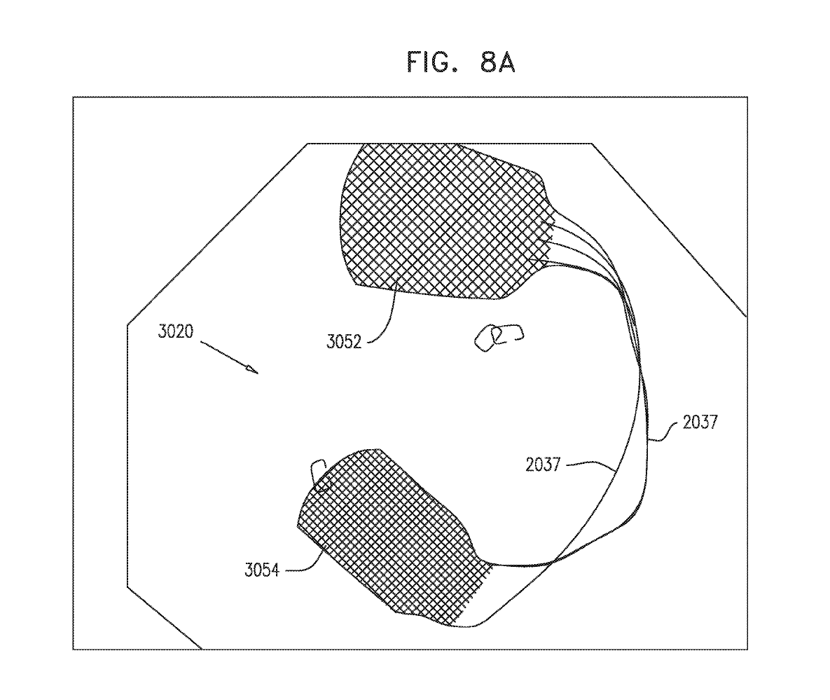

Reference is now made to FIGS. 1-7. As described hereinabove, some applications of the present invention comprise separate first and second stent anchors that are connected by a resilient connecting element to facilitate anchoring of the secretion-diversion guide within the gastrointestinal tract. Typically, this configuration allows for a diameter of a given stent anchor to remain generally unaffected while the other stent anchor is being squeezed due to a peristaltic wave applied to the other stent anchor. As shown by way of illustration in FIG. 7, first stent anchor 4052 and second stent anchor 4054 are arranged such that application of a squeezing force to second stent anchor 4054 generally does not cause a change in a diameter of pyloric portion 4252 and in a diameter of intragastric portion 4152 of anchor 4052. For example, application of a squeezing force to second stent anchor 4054 that reduces a diameter thereof by 50% (from D9, (FIG. 5A), to D9′ (FIG. 7)), does not produce an increase in a maximum diameter of proximal intragastric portion 4152 of more than 10% or a reduction in diameter D8 of pyloric portion 4252 of more than 10%. For some applications, application of a squeezing force to second stent anchor 4054 does not cause a change in a diameter of intragastric portion 4152 or of pyloric portion 4252. Accordingly, a peristaltic wave that causes squeezing of second stent anchor 4054 generally does not affect first stent anchor 4052, or the anchoring provided by the first stent anchor. Similarly, a peristaltic wave that causes squeezing of first stent anchor 4052 generally does not affect the diameter or consequent anchoring of second stent anchor 4054 (as shown in FIG. 5C).

It is noted that FIG. 7 shows apparatus 4020 by way of illustration and not limitation; the same concept described with reference to FIG. 7 applies to apparatus 2020, 3020 and 5020.

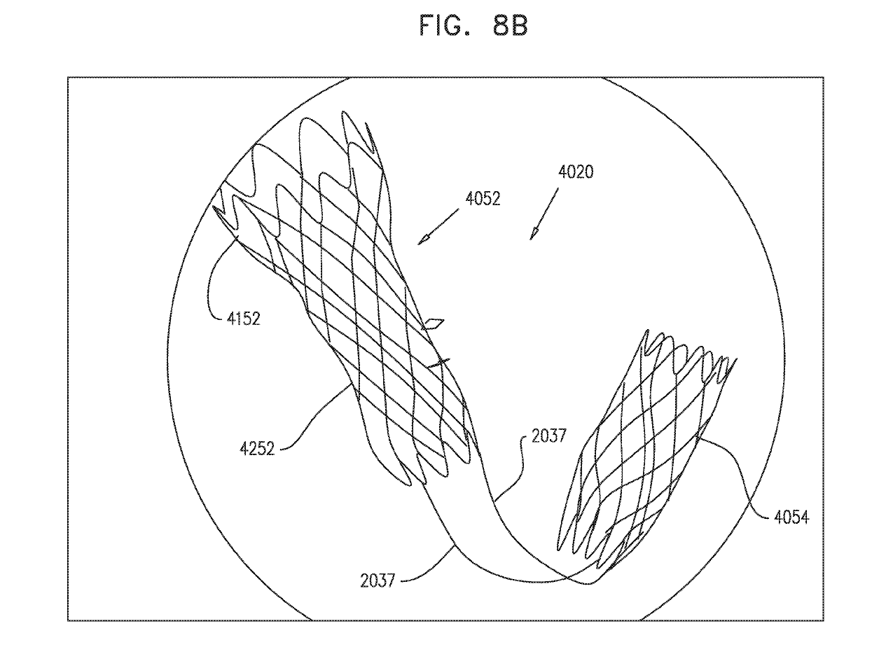

Reference is made to FIGS. 8A-B, which are reproductions of x-ray images acquired during experiments performed in accordance with some applications of the present invention. In these experiments, anchoring components of apparatus described herein were implanted in the gastrointestinal tract of a pig in order to test the stability thereof. As shown in FIGS. 8A-B and described hereinbelow, the implanted components remained generally stationary in the implantation sites and did not migrate distally in the gastrointestinal tract.

Reference is first made to FIG. 8A. In a first experiment, anchoring components (i.e., first and second stent anchors connected by two resilient connecting elements) of apparatus 3020 were implanted in a 70 kg swine (Sus scrofa domesticus). First and second stent anchors (3052 and 3054) connected by two resilient connecting elements (2037) were covered by a 10 mm diameter polyurethane sheath and inserted into the gastrointestinal tract of the pig through an incision in the stomach. First stent anchor 3052 was implanted entirely in the stomach (in the gastric antrum), and second stent anchor 3054 was implanted in the small intestine. Clips that are visible under fluoroscopy were attached to the pylorus and to the site of implantation in the small intestine. FIG. 8A shows a reproduction of an x-ray image taken four weeks post implantation. As shown, anchor 3052 remained within the stomach and did not pass through the pylorus. (The anchor is shown to be located above the clips that were attached to the pylorus.) Second anchor 3054 is shown to be located in the site of implantation in the small intestine to which the clip was attached.

Reference is now made to FIG. 8B. In a second experiment, anchoring components (i.e., first and second stent anchors connected by two resilient connecting elements) of apparatus 4020 were implanted in another 70 kg swine (Sus scrofa domesticus). First and second stent anchors (4052 and 4054) connected by two resilient connecting elements 2037 were covered by a 10 mm diameter polyurethane sheath and inserted into the gastrointestinal tract of the pig through an incision in the stomach. Intragastric portion 4152 of first stent anchor 4052 was implanted in the stomach (in the gastric antrum) and pyloric portion 4252 of first stent anchor 4052 was implanted in the pylorus. Second stent anchor 4054 was implanted in the small intestine. Clips that are visible by fluoroscopy based x-ray imaging were attached to the pylorus. FIG. 8B shows a reproduction of an x-ray image taken four weeks post implantation. As shown, intragastric portion 4152 of first stent anchor 4052 remained within the stomach and did not pass through the pylorus. (The anchor is shown to be located above the clips that were attached to the pylorus.) Pyloric portion 4252 of first stent anchor 4052 remained in the pylorus.

Therefore, results of both experiments shown in FIGS. 8A-B indicate that first and second stent anchors connected by two resilient connecting elements remain generally stationary in the implantation sites and do not migrate distally in the gastrointestinal tract. It is noted that the implants remained implanted in the pigs for more than three months, until the pigs were sacrificed.

Reference is made to FIGS. 1-8. For some applications, the pancreaticobiliary secretion-diversion guide affects hormonal secretion and/or action. Many hormones and enzymes participate in the digestion process, some of which are released by the gastrointestinal tract, for example, (a) GIP (Glucose-dependent insulinotropic peptide), a digestive hormone which is synthesized by intestinal K cells, and (b) GLP-1 (Glucagon-like peptide-1), which is synthesized by intestinal L cells. These hormones typically play a role in the regulation of the digestive process, and may affect fat metabolism and insulin secretion and action. Some applications of the present invention may modify the secretion and/or the action of these hormones and consequently modify the digestive process (including but not limited to fat metabolism and insulin secretion and action). For example, diversion of bile secretions to a distal location in the intestine, may stimulate GLP-1 secretion by intestinal L cells.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1-16. (canceled)

17. Apparatus for use with pancreaticobiliary secretions that enter a gastrointestinal tract of a subject at an anatomical entry location, the apparatus comprising:
a pancreaticobiliary secretion-diversion guide, comprising:
first and second stent anchors coupled to proximal and distal portions of the pancreaticobiliary secretion-diversion guide respectively, and configured to apply pressure to the wall of the gastrointestinal tract in order to reduce motion of the pancreaticobiliary secretion-diversion guide within the gastrointestinal tract, the first stent anchor comprising a proximal intragastric portion and a distal pyloric portion, the proximal intragastric portion (a) having a maximum unconstrained diameter of 45-65 mm, when no forces are applied thereto, and (b) being configured to diverge in a distal to proximal direction to a maximum diverged diameter of 50-60 mm, when a squeezing force is applied to the distal pyloric portion;
at least one resilient connecting element, each coupled to the first and second stent anchors and having a maximum diameter of 0.3-1.5 mm;
a collection-facilitation tube between the first and second stent anchors and disposed in contact with and around the at least one resilient connecting element, the collection-facilitation tube having an inner surface and an outer surface, the inner surface defining a lumen for passage of food therethrough, the outer surface disposed to inhibit contact of the secretions with food within the lumen and configured to define a space between the outer surface and the gastrointestinal tract for collecting the secretions;
a diversion tube, configured to:
be in fluid communication with the space between the outer surface and the gastrointestinal tract,
pass pancreaticobiliary secretions from the space to a location in the gastrointestinal tract that is distal to the anatomical entry location, and inhibit contact of the pancreaticobiliary secretions with food that has passed through the lumen.

18. The apparatus according to claim 17, wherein the first and second stent anchors are arranged such that application of a squeezing force to the second stent anchor that reduces a diameter thereof by 50% does not produce an increase in a maximum diameter of the proximal intragastric portion of more than 10%.

19. The apparatus according to claim 18, wherein the first stent anchor is arranged such that application of a squeezing force to the distal pyloric portion that reduces a diameter thereof by 50% causes an increase in the maximum diverged diameter the proximal intragastric portion by at least 1%.

20. The apparatus according to claim 17, wherein the first and second stent anchors are arranged such that application of a squeezing force to the second stent anchor that reduces a diameter thereof by 50% does not produce an increase in a maximum diameter of the proximal intragastric portion.

21. The apparatus according to claim 17, wherein the second anchor has a diameter of 20-40 mm when unconstrained.

22. The apparatus according to claim 17, wherein the pyloric portion of the first anchor has a diameter of 30-40 mm when unconstrained.

23. The apparatus according to claim 17, wherein the pyloric portion of the first anchor has a length of 10-30 mm when unconstrained, measured along a longitudinal axis of the anchor.

24. The apparatus according to claim 17, wherein the second anchor has a length of 40-60 mm when unconstrained, measured along a longitudinal axis of the anchor.

25. The apparatus according to claim 17, wherein the first anchor has a diameter of 20-40 mm when unconstrained.

26. The apparatus according to claim 17, wherein the intragastric portion of the first anchor has a length of 40-60 mm when unconstrained, measured along a longitudinal axis of the anchor.

27. The apparatus according to claim 17, wherein the diversion tube has a diameter of 4-20 mm.

28. The apparatus according to claim 17, wherein the collection-facilitation tube has a diameter of 20-25 mm.

29. The apparatus according to claim 17, wherein the anatomical entry location includes a duodenal papilla of the subject, and wherein the pancreaticobiliary secretion-diversion guide is configured to collect the pancreaticobiliary secretions that are secreted from the duodenal papilla.

30. The apparatus according to claim 29, wherein the pancreaticobiliary secretion-diversion guide is configured to collect the pancreaticobiliary secretions that are secreted from the duodenal papilla without entering a duct through which the secretions pass.

31. The apparatus according to claim 17, wherein the at least one resilient connecting element comprises at least two resilient connecting elements.

32. The apparatus according to claim 17, wherein the at least one resilient connecting element comprises exactly two resilient connecting elements.

33. The apparatus according to claim 17, wherein the at least one resilient connecting element comprise not more than eight resilient connecting elements.

34. The apparatus according to claim 17, wherein each resilient connecting element has a length of 10-18 cm when unconstrained, measured along a longitudinal axis of the resilient connecting element.

35. The apparatus according to claim 34, wherein each resilient connecting element has a length of 12-15 cm when unconstrained, measured along the longitudinal axis of the resilient connecting element.

36-64. (canceled)
